(19) United States
(12) Patent Application Publication
(10) Pub. No.: US 2003/0181829 A1
(43) Pub. Date: Sep. 25, 2003

(54) Anatomical dimension capture and delivery method

(76) Inventor: Jeffrey D. Edwards, Claremont, WA (US)

Correspondence Address: EDELL, SHAPIRO, FINNAN & LYTLE, LLC, 1901 RESEARCH BOULEVARD, SUITE 400, ROCKVILLE, MD, 20850, US

(21) Appl. No.: 10/221,750

(22) PCT Filed: Mar. 16, 2001
(86) PCT No.: PCT/AU01/00300

(51) Int. Cl. A61B 5/103; A61B 5/117

(52) U.S. Cl. 600/587

(57) ABSTRACT

In order to facilitate the capture of the three-dimensional anatomical measurements the present invention provides a self-adhesive label (10) having a plurality of visual markers providing thereon. The label (10) is sufficiently flexible to conform to a contour of an anatomical region to which it is adhered. The self-adhesive label is “T-shaped” and includes a first horizontal segment (12) having a series of evenly spaced, dark bands 14 separated by light spaces printed thereon. The label (10) may be manufactured from paper or a suitable plastics material and in this case is designed to be adhered to the lower or upper leg region of a patient with the first segment (12) wrapped partly around the leg so as to adopt the contours of the leg region. When a two-dimensional image of the anatomical region with the label (10) adhered thereto is made, variations in the width of the dark bands (14) and the light spaces, when viewed in the two-dimensional image, will provide an indication of fine variations in the contours of the leg region. A method of capturing three-dimensional anatomical measurements for use in manufacturing a customised therapeutic device is also described, as well as a method of producing a customised therapeutic device that involves transmitting information to a manufacturing facility via the Internet.

### FIELD OF INVENTION

The present invention relates to a means and method for enabling the capture of three dimensional anatomical measurements and relates particularly, though not exclusively, to such a means and method to facilitate the manufacture of customised therapeutic devices.

The term “therapeutic device”as used throughout this specification is intended to cover orthopaedic braces, splints and other devices which can be used either for therapeutic treatment or preventative protection of anatomical regions and structures of the human body.

### BACKGROUND TO THE INVENTION

It is desirable in many medical conditions to provide patients with supporting or therapeutic devices that are specifically designed to their individual needs. For example, with conditions affecting the knee such as osteo-arthritis and anterior cruciate ligament injuries a custom-made oritopaedic brace provides superior fit, comfort and therapeutic benefit compared to off-the-shelf devices.

The process of measuring a patient for such custom-made devices has traditionally involved either the taking of a plaster cast or the recording of anatomical mesurements using rules or tapes. In the case of plaster casts, the patient is required to remain stationary to allow the plaster cast to set, and is often required to adopt a non-load-bearing stance to allow access to the entire anatomical region. In the case of manual measurements using rules and tapes, the patient may be required to change stance to allow the various measurements to be taken. As the patient may adopt a load-bearing stance during some stages of measurement, and only partially- or non-load-bearing during others, the accuracy of the measurements is questionable.

The profile of the musculature of the knee and the relative angle of the skeleton in relation to that musculature, changes significantly during articulation. Hence, any measurements taken in a stance other than fill dynamic load-bearing, will yield a model with inherent dimensional inaccuracies. Traditionally, measurements taken using tapes or rules do not provide important information relating to the circumferential shape of the anatomical region, which is essential for an accurate fit. Although plaster casts may provide some of this additional information, this method involves significant additional costs and time delays due to high transport costs and significant manual handling at the manufacturing site required to extract a facsimile from the original cast.

Neither plaster casts nor manual measuring systems provide a sufficiently accurate, convenient or cost and time-effective manner of capturing and delivering the anatomical information required to achieve a good therapeutic outcome in the production of custom made therapeutic devices.

### SUMMARY OF THE INVENTION

The present invention was developed with a view to providing a method and means of capturing and delivering anatomical measurements that provide an accurate representation of the dynamic musculature profile during normal articulation.

Throughout this specification the term “comprising” is used inclusively, in the sense that there may be other features and/or steps included in the invention not expressly defined or comprehended in the features or steps subsequently defined or described. What such other features and/or steps may include will be apparent from the specification read as a whole.

According to one aspect of the present invention there is provided a dimensional scaling means for enabling the capture of three-dimensional anatomical measurements, the dimensional scaling means comprising:

a flexible planar member having a plurality of visual markers provided thereon at predetermined intervals, the planar member being capable of conforming to a contour of an anatomical region, whereby, in use, when said planar member is placed adjacent said anatomical region such that said planar member conforms to the contour of said anatomical region and a two-dimensional image of said anatomical region is generated, the two-dimensional image provides three-dimensional information concerning the shape of said anatomical region.

Preferably said flexible planar member is in the form of a self-adhesive label having said visual markers printed thereon at predetermined intervals. In one preferred form of the invention said self-adhesive label is “T-shaped” and is adapted to be adhered to the lower or upper leg region of a patient. Preferably the self-adhesive label comprises a first horizontal segment adapted to wrap partly around the leg and adopt the contours of said leg region, said first segment having a series of evenly spaced, dark bands separated by light spaces printed hereon whereby, in use, variations in the width of the dark bands and the light spaces when viewed in said two dimensional image provide an indication of fine variations in the contours of said leg region

Preferably said “T-shaped” label has a second vertical segment adapted to adhere to the thigh or shin of the leg, said second segment having two visual makers separated by a predetermined distance, one of said visual markers being in the form of an optical-cross hair point. Preferably, in use, the “T-shaped” label is adhered to the leg with said cross hair point located over the kneecap.

According to another aspect of the present invention, there is provided a method of capturing three-dimensional anatomical measurements for use in manufacturing a customised therapeutic device, the method comprising:

adhering a dimensional scaling means to an anatomical region for which a therapeutic device is to be manufactured, said dimensional scaling means comprising a flexible planar member having a plurality of visual markers provided thereon and adapted to conform to a contour of the anatomical region;

producing a two-dimensional image of the anatomical region with said dimensional scaling means adhered thereto; and

converting said two-dimensional image into a format suitable for transmission to a manufacturing facility.

Preferably said step of producing a two-dimensional image is effected using a digital camera.

According to a still further aspect of the present invention there is provided a method of producing a customised therapeutic device for a patient, the method comprising:

producing one or more images of an anatomical region of the patient requiring a therapeutic device;

converting said one or more images into a format suitable for transmission to a manufacturing facility;

transmitting said converted images and any additional information required to the manufacturing facility via a computer network;

receiving said converted images and any additional information and converting into anatomical information suitable for use in manufacturing a therapeutic device; and

manufacturing the therapeutic device based on said anatomical information.

Advantageously said computer network is the internet.

### BRIEF DESCRIPTION OF DRAWINGS

In order to facilitate a more detailed understanding of the nature of the invention, a preferred embodiment of the means and method of capturing three-dimensional anatomical measurements will now be described in detail, by way of example only, with reference to the accompanying drawings, in which:

FIG. 1 illustrates a preferred embodiment of a dimensional scaling means in the form of a self-adhesive label;

FIG. 2 illustrates a preferred embodiment of a method of producing a customised therapeutic device in accordance with the present invention.

### DETAILED DESCRIPTION OF PREFERRED EMBODIMENT

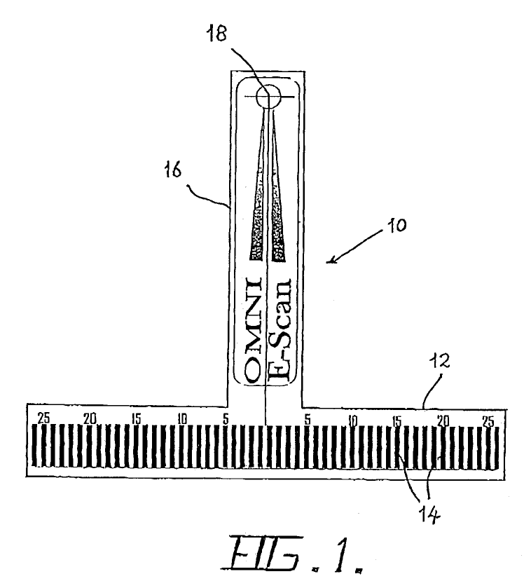

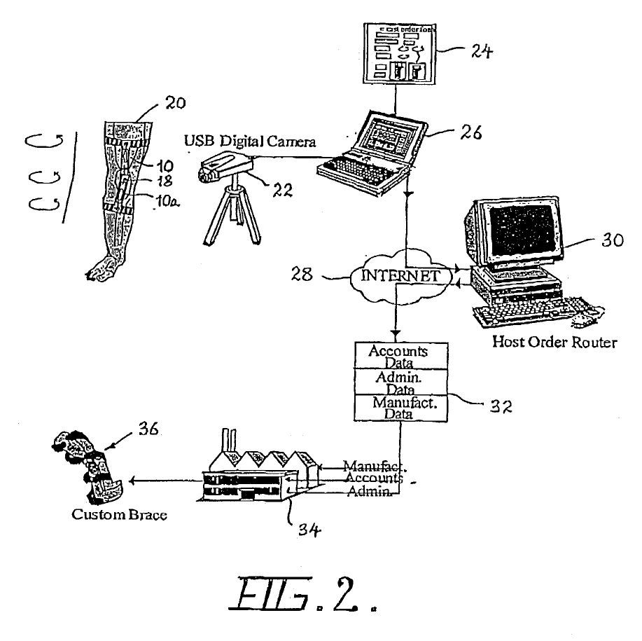

In order to facilitate the capture of three-dimensional anatomical measurements the present invention provides a dimensional scaling means comprising a flexible planar member having a plurality of visual markers provided thereon. FIG. 1 illustrates a preferred embodiment of such a dimensional scaling means in the form of a self-adhesive label 10 which is sufficiently flexible to conform to a contour of an anatomical region to which it is adhered. In this embodiment, the self-adhesive label is “T-shaped” and comprises a first horizontal segment 12 having a series of evenly space dark bands 14 separated by light spaces printed thereon. The label 10 may be manufactured from paper or a suitable plastics material and in this case is adapted to be adhered to the lower or upper leg region of a patient with the first segment 12 wrapped partly around the leg so as to adopt the contours of the leg region (see FIG. 2). When a two-dimensional image of the anatomical region with the label 10 adhered thereto is made, variations in the width of the dark bands 14 and the light spaces when viewed in the two-dimension image will provide an indication of fine variations in the contours of the leg region.

In this embodiment the self-adhesive label 10 also includes a second vertical segment 16 adapted to adhere to the front of the thigh or shin of the leg. The second segment 16 has two visual makers separated by a predetermined distance one of said visual markers being in the form of an optical-cross hair point 18. In the illustrated example, the known distance between the cross hair point 18 and the lower edge of the bands 14 is exactly 200millimetres. Hence, provided that this segment of the label 10 is adhered to a relatively flat region of the anatomical structure, it provides an optical reference that can be used to scale other anatomical dimensions that are visible in the two-dimensional. When adhered to the knee, the cross hair point 18 is preferably located directly over the mid-patella point, which provides a reference point for all other anatomical dimensions extracted from the two-dimensional image.

A method of capturing three-dimensional anatomical measurements and delivering such measurements for use in manufacturing a customised therapeutic device will now be described with reference to FIG. 2.

For the purposes of illustration, the manufacture of a customised knee brace will be described, however it will be apparent that substantially the same method can be applied to the manufacture of therapeutic devices for other anatomical regions of the human body. Two dimensional scaling means in the form of adhesive labels 10 are adhered to the leg 20 above and below the knee as shown in FIG. 2. A first label 10 has the horizontal segment 12 partly wrapped around the thigh of the leg 20, whereas a second label 10a has its horizontal segment 12a partly wrapped around the shin of the leg. The cross hair points 18 on both labels 10, 10a overlap above the mid-patella point of the kneecap.

A two-dimensional image of the leg is produced with the dimensional scaling means 10, 10a adhered thereto. In the illustrated embodiment, a USB digital camera 22 is employed to produce the two-dimensional image. The digital data representing the two-dimensional image is then converted into a format suitable for transmission to a manufacturing facility. For example, the digital data may be compressed using the J-PEG format and incorporated into an E-Cast Order Form 24 using a proprietary software program provided on the end-user's laptop computer 26. This order form may be downloaded via a computer network, such as the Internet 28 from a website provided on a host order router or server 30 operated by the manufacturer or an intermediary service provider. When the order form 24 has been completed it is retransmitted to the host order server 30 where it is combined with accounts data and administration date, including the name, address and account details of the person or organisation placing the order, This information is then transmitted to the manufacturer 32 which is able to use the anatomical data to produce a customised leg brace 36.

The anatomical manufacturing data may be produced entirely automatically using proprietary software based on the two dimensional image data captured using the USB. digital camera 22. This two dimensional image data is employed to produce three dimensional anatomical measurements based on the additional information provided by the dimensional scaling means. This information includes length and width, circumference and an indication of fine variations in the contours of the shin and thigh regions. his information is processed to produce a virtual wire-frame model of the leg from which a virtual cast of the leg can be produced. The software is able to select suitable standard components from an inventory of various standard sized shells for leg braces. For example, based on the three-dimensional anatomical measurements derived, it may select a No. 4 lower shell and a No. 2 upper shell to construct a customised leg brace.

Further modifications may be made to the standard shells so that they conform more closely to the contours of the patient's leg. The actual process of constructing the leg brace from standard components may also be automated, so that there is no need for manual intervention at any stage of the manufacturing process. Alternatively the leg brace may be assembled manually at the manufacturing facility. The completed customised brace can then be transported back to the address of the person or organisation which submitted the E-Cast Order Form.

It will be seen that this method of producing a customised therapeutic device for a patient lends itself to the placing of an order for a customised device by persons with very little prior training. Therefore, a sales assistant in a pharmacy or a nursing assistant in a doctor's surgery or physiotherapist's clinic can readily place an order for a customised therapeutic device and provide the patient with the device within a matter of days.

From the above description of a preferred embodiment of the means and method of enabling the capture of three-dimensional anatomical measurements to facilitate the manufacture of customised therapeutic devices, it will be evident it has a number of significant advantages over current techniques, including the following advantages:

(i) it enables three-dimensional anatomical measurements to be captured using a two-dimensional image produced by a standard digital camera;

(ii) it enables accurate anatomical measurements to be obtained easily and rapidly with the patient in a full dynamic load-bearing stance;

(iii) it enables the electronic lodgement of an order for a customised therapeutic device via the internet;

(iv) it significantly reduces the time required to manufacture a customised therapeutic device and lends itself to full automation; and,

(v) it substantially reduces the costs of producing a customised therapeutic device as it eliminates the need for manual measurements or the production of a plaster cast of the anatomical region.

Numerous variations and modifications will suggest themselves to persons skilled in the orthopaedic and theapeutic arts, in addition to those already describe without department from tie basic inventive concepts. For example, the dimensional scaling means need not be in the form of a self-adhesive label. Furthermore, the dimensional scaling means may be of any suitable shape depending on the anatomical region for which three-dimensional anatomical measurements arc required to be captured. All such variations and modifications are to be considered within the scope of the present invention, the nature of which is to be determined from the foregoing description and the appended claims

The claims defining the invention

1. A dimensional scaling means for enabling the capture of three-dimensional anatomical measurements, the dimensional scaling means comprising:
a flexible planar member having a plurality of visual markers provided thereon at predetermined intervals, said planar member being capable of conforming to a contour of an anatomical region, whereby, in use, when said planar member is placed adjacent said anatomical region such that said planar member conforms to the contour of said anatomical region and a two-dimensional image of said anatomical region is generated, the two-dimensional image provides three-dimensional information concerning the shape of said anatomical region.

2. A dimensional scaling means as defined in claim 1, wherein said flexible planar member is in the form of a self-adhesive label having said visual markers printed thereon.

3. A dimensional scaling means as defined in claim 2, wherein said self-adhesive label is “T-shaped” and is adapted to be adhered to the lower or upper leg region of a patient.

4. A dimensional scaling means as defined in claim 3, wherein the self-adhesive label comprises a first horizontal segment adapted to wrap partly around the leg and adopt the contours of said leg region, said first segment having a series of evenly spaced, dark bands separated by light spaces printed thereon whereby, in use, variations in the width of the dark bands and the light spaces when viewed in said two-dimensional image provide an indication of fine variations in the contours of said leg region.

5. A dimensional scaling means as defined in claim 4, wherein said “T-shaped” label has a second vertical segment adapted to adhere to the thigh or shin of the leg, said second segment having two visual markers separated by a predetermined distance, one of said visual markers being in the form of an optical-cross hair point.

6. A method of capturing three-dimensional anatomical measurements for use in manufacturing a customised therapeutic device, the method comprising:
adhering a dimensional scaling means to an anatomical region for which a therapeutic device is to be manufactured, said dimensional scaling means comprising a flexible planar member having a plurality of visual markers provided thereon and adapted to conform to a contour of the anatomical region;
producing a two-dimensional image of the anatomical region with said dimensional scaling means adhered thereto; and
converting said two-dimensional image into a format suitable for transmission to a manufacturing facility.

7. A method of capturing three dimensional anatomical measurements as defined in claim 6, wherein said step of producing a two-dimensional image is effected using a digital camera.

8. A method of capturing three-dimensional anatomical measurements as defined in claim 7, wherein said step of converting the two-dimensional image involves converting it into an electronic format suitable for transmission over a computer network.

9. A method of capturing three-dimensional anatomical measurements as defined in claim 8, wherein said flexible planar member is in the form of a self-adhesive label having said visual makers printed thereon at predetermined intervals and said step of adhering the dimensional scaling means involves g the self-adhesive label to the anatomical region.

10. A method of capping three dimensional anatomical measurements as defined in claim 9, wherein the self-adhesive label comprises a first segment, and said step of affixing the self-adhesive label to the anatomical region involves wrapping the first segment around the anatomical region so that it adopts the contours of said anatomical region, said first segment having a sends, of evenly spaced, dark bands separated by light spaces printed thereon whereby, in use, variations in the width of the dark bands and the light spaces when viewed in said two-dimensional image provide an indication of fine variations in the contours of said anatomical region.

11. A method of capturing three-dimensional anatomical measurements as defined in claim 10, wherein said label has a second segment adapted to adhere to the anatomical region, said second segment having two visual markers Separated by a predetermined distance, one of said visual markers being in the form of an optical-cross hair point.

12. A method of capturing dimensional anatomical measurements as defined in claim 11, wherein the label is adhered to a leg with said cross hair point located over the kneecap.

13. A method of producing a customised therapeutic device for a patient, the method comprising:
producing one or more images of an anatomical region of the patient requiring a therapeutic device;
converting said one or more images into a format suitable for transmission to a manufacturing facility;
transmitting said converted images and any additional information required to the manufacturing facility via a computer network;
receiving said converted images and any additional information and converting into anatomical information suitable for use in manufacturing a therapeutic device; and
manufacturing the therapeutic device bed on said anatomical information.

14. A method of producing a customised therapeutic device as defined in claim 13, wherein said computer network is the Internet.

15. A method of producing a customised therapeutic device as defined in claim 14, wherein said step of manufacturing involves using said anatomical information to produce a virtual wire-frame model of the anatomical region from which a virtual cast of the anatomical region can be produced.

16. A method of producing a customised therapeutic device as defined in claim 15, wherein said step of manufacturing further involves select suitable standard components from an inventory of various standard sized shells for the therapeutic device.
